(19) United States
(12) Patent Application Publication
(10) Pub. No.: US 2017/0037275 A1
(43) Pub. Date: Feb. 9, 2017

(54) HOOF REPAIR COATING

(71) Applicant: DM Healthcare Products, Inc., San Diego, CA (US)

(72) Inventor: ALLEN D. JOHNSTON, San Diego, CA (US)

(21) Appl. No.: 15/231,743

(22) Filed: Aug. 8, 2016

Related U.S. Application Data
(60) Provisional application No. 62/202,817, filed on Aug. 8, 2015.

(51) Int. Cl. C09D 135/02; C08K 3/08; A61K 31/785; A01L 15/00; A61K 9/00; C08K 3/36; C08K 5/13

(52) U.S. Cl. CPC C09D 135/02; C08K 3/36; C08K 3/08; C08K 5/13; A01L 15/00; A61K 9/0017; A61K 31/785; C08K 2003/0806

(57) ABSTRACT

The present invention relates to curable coatings for treatment and repair of the ungulate hoof. These coatings are very easy to apply to the hoof of the animal, and cure to a tack-free hard coating as the animal walks around in direct sunlight or upon short exposure to ultraviolet light. Regular treatment of the coating to the hoof prevents hoof cracks in the long run and improves the health of the animal.

### RELATED APPLICATION

This application claims the benefit of priority to U.S. Provisional Application Ser. No. 62/202,817, filed Aug. 8, 2015, the entire disclosure of which is incorporated herein by reference.

### FIELD OF INVENTION

The present invention relates to ungulate hoof repair and maintenance methods and compositions. In particular, the present invention relates to multiple, easy to apply, light-curable formulations for use in repairing and protecting horse hooves.

### BACKGROUND OF INVENTION

For thousands of years human beings have depended on domesticated animals such as cattle and horses to help us get more work done. These ungulate mammals have much more muscle power than human beings do and can get much more work done than humans can. Horses have especially been an indispensable part of human civilization.

Thousands of years ago, the horse was domesticated, taken out of its natural habitat, its diet was changed, and it was put to work by humans on a variety of surfaces. It was at this time that the farrier profession was born to care for the equine hoof. The main job of the farrier is trimming and shoeing of horse hooves. The trimming and proper shoeing of the horse hoof is the best way to protect the equine foot from undue wear and injury, although it has also been found that a proper nutrition for the animal is extremely important to having larger and stronger hooves.

The tip of the toe of an ungulate mammal is called the “hoof”. The wall of the hoof is composed of a tough keratin covering, similar to the human nail. Also similar to the human nail, the hoof is continuously growing and being worn down by use.

As the major weight-bearing structure of the horse's hoof, the hoof wall is under tremendous stress. The stress can be caused by numerous factors, including gait, weight, exercise, nutrition, as well as many environmental factors. Damage to the hoof wall can cause problems for the horse such as quarter cracks, toe cracks, crushed heels, voids, and thin brittle walls.

In recent decades, curable resins have come into use in protecting and repairing the horse hoof wall. These materials are often based on acrylics or reactive urethanes. There are several drawbacks associated with these materials, including: lack of strength; difficulty of use; poor work life, and toxicity.

The acrylic formulations are often cured via the decomposition of peroxide with an amine activator. The strength and durability of these compounds often comes through applying a polyester fabric, followed by the acrylic formulation, thereby forming a high strength composite. The application of such materials is very difficult and time consuming, and suppliers of such products recommend that a highly experienced farrier apply them.

The reactive urethane formulations are based on the reaction of a diisocyanate with a polyol in a two-part formulation. In this type of application, a mixing gun is typically used to mix the portions of the materials together and apply them to the hoof of an animal. This type of system has some significant shortcomings as well. For example the use of a static mixer or mixing gun can be very expensive. Once the two components are mixed together, the polyurethane formation is very fast uncontrollable) and produces a great deal of heat, which can be uncomfortable for the animal. Furthermore, the non-uniform layer of polyurethane that is deposited on the hoof must be abraded and shaped to a uniform coating. The abrading and shaping requires a great deal of time even by a very experienced farrier, during which time, the animal is stressed.

There is thus a need for hoof coating products that anyone could easily applied to the hoof, that are non-irritating, that produce a non-tacky surface rapidly, that provide protection to the hoof, and that are aesthetically pleasing.

### SUMMARY OF INVENTION

Easy to apply formulations have been developed to be used as coatings for repairing and or preventing damage to the equine hoof. The light-curable invention formulation compositions include: a) at least one acrylated urethane; b) at least one adhesion promoter(s); c) at least one tertiary amine; d) at least one filler; e) at least one photoinitiator; and at least one free radical inhibitors, where the composition is suitable for application to the hoof of ungulate animals. In certain embodiments, the compositions also include pigments.

The at least one acrylated urethane can be, for example, selected from:

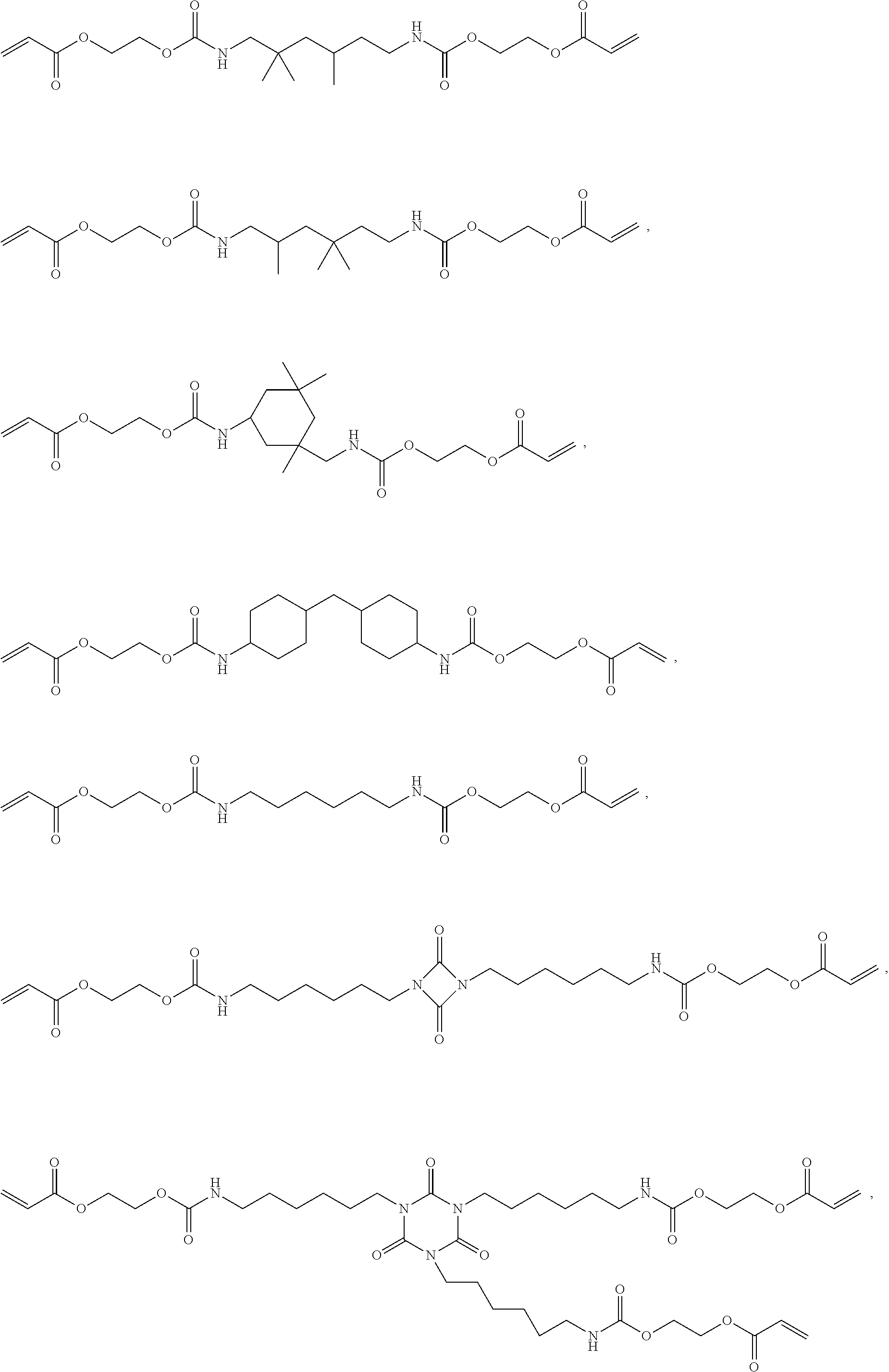

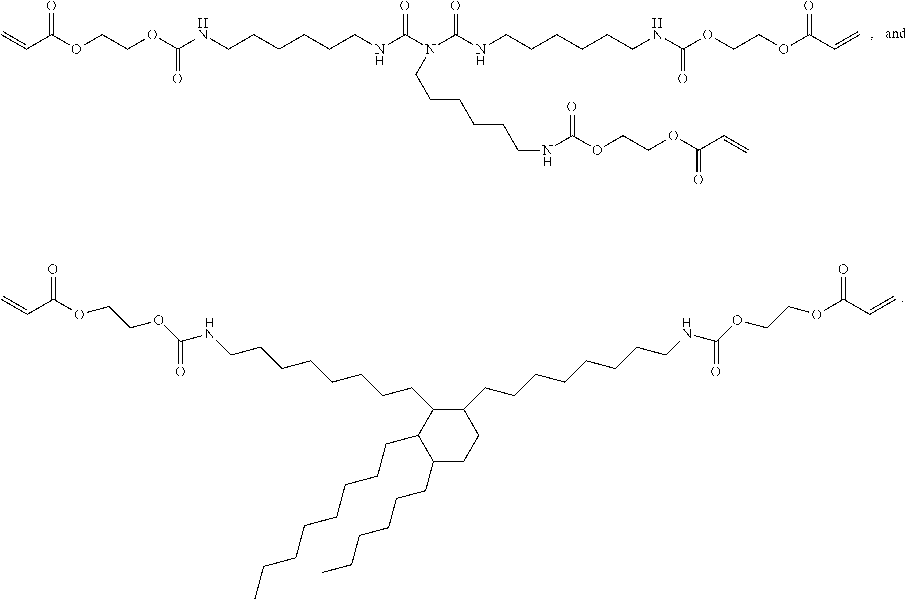

In certain aspects, the at least one acrylated urethane comprises about 55 to about 95 weight percent (wt %) based on the total weight of the composition. In other aspects at least one acrylated urethane comprises about 65 to 85-wt % based on the total weight of the composition.

In various embodiments of the invention, the at least one adhesion promoter is 2-hydroxyethyl acrylate phthalate and/or 2-hydroxyethyl acrylate. Typically, the at least one adhesion promoter comprises 0.5 to about 30 wt % based on the total weight of the composition. In certain embodiments, the at least one adhesion promoter comprises 10 to about 25 wt % based on the total weight of the composition.

The at least one filler can, for example, comprise silica. Typically, the at least one filler comprises 0.9 to about 3.5 wt % based on the total weight of the composition.

In various embodiments of the invention, the at least one photoinitiator comprises ethyl(2,4,6-trimethylbenzoyl)phenyl phosphonate and/or 2,4,6-trimethylbenzoyldiphenyl phosphine oxide. Typically, the at least one photoinitiator comprises 3 to about 10 wt % based on the total weight of the composition.

In certain embodiments of the invention, the at least one tertiary amine comprises ethyl 4-dimethylaminobenzoate. Typically, the at least one tertiary amine comprises 2 to about 6 wt % based on the total weight of the composition.

In one aspect of the invention, the at least one free radical inhibitor includes butylated hydroxytoluene.

Advantageously, certain compositions of the invention are thixotropic, which may facilitate application. Typically, the coating compositions of the invention cure to a hard, tack-free coating upon exposure to direct sunlight or ultraviolet light after brushing onto the surface of the hoof of the ungulate.

The advantages of this system is that it is extremely operator friendly, and the user (e.g. farrier) has ultimate control over the cure of the material.

The current hoof coatings are reactive systems intended to repair deficiencies in the hoof wall. In contrast, the invention compositions are prophylactic in nature: periodic applications of the material along with normal trimming and cleaning of the hoof prevents cracks from forming and lead to better overall health for the animal.

The compositions of the invention have a very long work life when kept out of direct sunlight. The farrier or operator applying the formulation can brush the material on to the hoof of the animal and make sure that it is evenly coated and smooth. As the animal walks around in sunlight the material cures to produce a hard tack-free coating in about one minute. Alternatively, the material can be cured very quickly within 30 seconds if the operator has access to a UV-light set to 380 nm.

Thus also provided by the invention are methods of protecting, repairing and/or decoratively embellishing the hoof of an ungulate animal comprising the steps of: (a) providing a composition as described herein; (b) brushing the composition on the surface of the hoof of the ungulate; and exposing the composition to direct sunlight or ultraviolet light; where the composition cures to a hard, tack-free coating upon exposure to direct sunlight or ultraviolet light, thereby protecting, repairing and/or decoratively the hoof of an ungulate animal.

### DETAILED DESCRIPTION

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. Furthermore, use of the term “including” as well as other forms, such as “includes,” and “included,” is not limiting.

The section headings used herein are for organizational purposes only and are not to be construed as limiting the subject matter described.

Unless specific definitions are provided, the nomenclatures utilized in connection with, and the laboratory procedures and techniques of analytical chemistry, synthetic organic and inorganic chemistry described herein are those known in the art, such as those set forth in “IUPAC Compendium of Chemical Terminology: IUPAC Recommendations (The Gold Book)” (McNaught ed.; International Union of Pure and Applied Chemistry, 2^{nd} Ed 1997) and “Compendium of Polymer Terminology and Nomenclature: IUPAC Recommendations 2008” (Jones et al., eds; International Union of Pure and Applied Chemistry, 2009). Standard chemical symbols are used interchangeably with the full names represented by such symbols. Thus, for example, the terms “hydrogen” and “H” are understood to have identical meaning. Standard techniques may be used for chemical syntheses, chemical analyses, and formulation.

### DEFINITIONS

“About” as used herein means that a number referred to as “about” comprises the recited number plus or minus 1-10% of that recited number. For example, “about” 100 degrees can mean 95-105 degrees or as few as 99-101 degrees depending on the situation. Whenever it appears herein, a numerical range such as “1 to 20” refers to each integer in the given range; e.g., “1 to 20 carbon atoms” means that an alkyl group can contain only 1 carbon atom, 2 carbon atoms, 3 carbon atoms, etc., up to and including 20 carbon atoms (although the term “alkyl” also includes instances where no numerical range of carbon atoms is designated).

“Adhesive” or “adhesive compound” as used herein, refers to any substance that can adhere or bond two items together. Implicit in the definition of an “adhesive composition” or “adhesive formulation” is the fact that the composition or formulation is a combination or mixture of more than one species, component or compound, which can include adhesive monomers, oligomers, and/or polymers along with other materials, whereas an “adhesive compound” refers to a single species, such as an adhesive polymer or oligomer.

More specifically, adhesive composition refers to un-cured mixtures in which the individual components in the mixture retain the chemical and physical characteristics of the original individual components of which the mixture is made. Adhesive compositions are typically malleable and may be liquids, paste, gel or another form that can be applied to an item so that it can be bonded to another item.

“Cured adhesive,” “cured adhesive composition” or “cured adhesive compound” refers to adhesives components and mixtures obtained from reactive curable original compound(s) or mixture(s) thereof which have undergone a chemical and/or physical changes such that the original compound(s) or mixture(s) is(are) transformed into a solid, substantially non-flowing material. A typical curing process may involve crosslinking.

“Curable” means that an original compound(s) or composition material(s) can be transformed into a solid, substantially non-flowing material by means of chemical reaction, crosslinking, radiation crosslinking, or the like. Thus, adhesive compositions of the invention are curable, but unless otherwise specified, the original compound(s) or composition material(s) is(are) not cured.

“Thermoplastic,” as used herein, refers to the ability of a compound, composition or other material (e.g. a plastic) to dissolve in a suitable solvent or to melt to a liquid when heated and freeze to a solid, often brittle and glassy, state when cooled sufficiently.

“Thermoset,” as used herein, refers to the ability of a compound, composition or other material to irreversibly “cure” resulting in a single three-dimensional network that has greater strength and less solubility compared to the non-cured product. Thermoset materials are typically polymers that may be cured, for example, through heat (e.g. above 200° Celsius), via a chemical reaction (e.g. epoxy ring-opening, free-radical polymerization, etc or through irradiation (e.g. visible light, UV light, electron beam radiation, ion-beam radiation, or X-ray irradiation).

Thermoset materials, such as thermoset polymers or resins, are typically liquid or malleable forms prior to curing, and therefore may be molded or shaped into their final form, and/or used as adhesives. Curing transforms the thermoset resin into a rigid infusible and insoluble solid or rubber by a cross-linking process. Thus, energy and/or catalysts are typically added that cause the molecular chains to react at chemically active sites (unsaturated or epoxy sites, for example), linking the polymer chains into a rigid, 3-D structure. The cross-linking process forms molecules with a higher molecular weight and resultant higher melting point. During the reaction, when the molecular weight of the polymer has increased to a point such that the melting point is higher than the surrounding ambient temperature, the polymer becomes a solid material.

“Cross-linking,” as used herein, refers to the attachment of two or more oligomer or longer polymer chains by bridges of an element, a molecular group, a compound, or another oligomer or polymer. Crosslinking may take place upon heating or exposure to light; some crosslinking processes may also occur at room temperature or a lower temperature. As cross-linking density is increased, the properties of a material can be changed from thermoplastic to thermosetting.

The term “monomer” refers to a molecule that can undergo polymerization or copolymerization thereby contributing constitutional units to the essential structure of a macromolecule (a polymer).

“Polymer” and “polymer compound” are used interchangeably herein, to refer generally to the combined the products of a single chemical polymerization reaction. Polymers are produced by combining monomer subunits into a covalently bonded chain. Polymers that contain only a single type of monomer are known as “homopolymers,” while polymers containing a mixture of monomers are known as “copolymers.”

As used herein, “aliphatic” refers to any alkyl, alkenyl, cycloalkyl, or cycloalkenyl moiety.

“Aromatic hydrocarbon” or “aromatic” as used herein, refers to compounds having one or more benzene rings.

“Alkane,” as used herein, refers to saturated straight-chain, branched or cyclic hydrocarbons having only single bonds. Alkanes have general formula CₙH₂ₙ₊₂.

“Cycloalkane,” refers to an alkane having one or more rings in its structure.

As used herein, “alkyl” refers to straight or branched chain hydrocarbyl groups having from 1 up to about 500 carbon atoms. “Lower alkyl” refers generally to alkyl groups having 1 to 6 carbon atoms. The terms “alkyl” and “substituted alkyl” include, respectively, substituted and unsubstituted C₁-C₅₀₀ straight chain saturated aliphatic hydrocarbon groups, substituted and unsubstituted C₂-C₂₀₀ straight chain unsaturated aliphatic hydrocarbon groups, substituted and unsubstituted C₄-C₁₀₀ branched saturated aliphatic hydrocarbon groups, substituted and unsubstituted C₁-C₅₀₀ branched unsaturated aliphatic hydrocarbon groups.

For example, the definition of “alkyl” includes but is not limited to: methyl (Me), ethyl (Et), propyl (Pr), butyl (Bu), pentyl, hexyl, heptyl, octyl, nonyl, decyl, undecyl, ethenyl, propenyl, butenyl, penentyl, hexenyl, heptenyl, octenyl, nonenyl, decenyl, undecenyl, isopropyl (i-Pr), isobutyl (i-Bu), tert-butyl (t-Bu), sec-butyl (s-Bu), isopentyl, neopentyl, cyclopropyl, cyclobutyl, cyclopentyl, cyclohexyl, cycloheptyl, cyclooctyl, cyclopentenyl, cyclohexenyl, cycloheptenyl, cyclooctenyl, methylcyclopropyl, ethylcyclohexenyl, butenylcyclopentyl, tricyclodecyl, adamantyl, norbornyl and the like.

“Substituted alkyl” refers to alkyl moieties bearing substituents that include but are not limited to alkyl, alkenyl, alkynyl, hydroxy, oxo, alkoxy, mercapto, cycloalkyl, substituted cycloalkyl, heterocyclic, substituted heterocyclic, aryl, substituted aryl (e.g., arylC₁₋₁₀alkyl or arylC₁₋₁₀alkyloxy), heteroaryl, substituted heteroaryl (e.g., heteroarylC₁₋₁₀alkyl), aryloxy, substituted aryloxy, halogen, haloalkyl (e.g., trihalomethyl), cyano, nitro, nitrone, amino, amido, carbamoyl, ═O, ═CH—, —C(O)H, —C(O)O—, —C(O)—, —S—, —S(O)₂, —OC(O)—O—, —NR—C(O), —NR—C(O)—NR, —OC(O)—NR, where R is H or lower alkyl, acyl, oxyacyl, carboxyl, carbamate, sulfonyl, sulfonamide, sulfuryl, C₁₋₁₀alkylthio, arylC₁₋₁₀alkylthio, C₁₋₁₀alkylamino, arylC₁₋₁₀alkylamino, N-aryl-N—C₁₋₁₀alkylamino, C₁₋₁₀alkyl carbonyl, arylC₁₋₁₀alkylcarbonyl, C₁₋₁₀alkylcarboxy, aryl C₁₋₁₀alkylcarboxy, C₁₋₁₀alkyl carbonylamino, aryl C₁₋₁₀alkylcarbonylamino, tetrahydrofuryl, morpholinyl, piperazinyl, and hydroxypyronyl.

As used herein, “cycloalkyl” refers to cyclic ring-containing groups containing in the range of about 3 up to about 20 carbon atoms, typically 3 to about 15 carbon atoms. In certain embodiments, cycloalkyl groups have in the range of about 4 up to about 12 carbon atoms, and in yet further embodiments, cycloalkyl groups have in the range of about 5 up to about 8 carbon atoms. and “substituted cycloalkyl” refers to cycloalkyl groups further bearing one or more substituents as set forth below.

As used herein, the term “aryl” represents an unsubstituted, mono-, di- or trisubstituted monocyclic, polycyclic, biaryl aromatic groups covalently attached at any ring position capable of forming a stable covalent bond, certain preferred points of attachment being apparent to those skilled in the art (e.g., 3-phenyl, 4-naphtyl and the like). The aryl substituents are independently selected from the group consisting of halo, —OH, —SH, —CN, —NO₂, trihalomethyl, hydroxypyronyl, C₁₋₁₀alkyl, arylC₁₋₁₀alkyl, C₁₋₁₀alkyloxyC₁₋₁₀alkyl, arylC₁₋₁₀alkyloxyC₁₋₁₀alkyl, C₁₋₁₀alkylthioC₁₋₁₀alkyl, arylC₁₋₁₀alkylthioC₁₋₁₀alkyl, C₁₋₁₀alkylaminoC₁₋₁₀alkyl, arylC₁₋₁₀alkylaminoC₁₋₁₀alkyl, N-aryl-N—C₁₋₁₀alkylaminoC₁₋₁₀alkyl, C₁₋₁₀alkylcarbonylC₁₋₁₀alkyl, aryl C₁₋₁₀alkylcarbonyl C₁₋₁₀alkyl, C₁₋₁₀alkylcarboxyC₁₋₁₀alkyl, arylC₁₋₁₀alkylcarboxyC₁₋₁₀alkyl, C₁₋₁₀alkylcarbonylaminoC₁₋₁₀alkyl, and arylC₁₋₁₀alkylcarbonylaminoC₁₋₁₀alkyl.

As used herein, “hetero” refers to groups or moieties containing one or more heteroatoms such as N, O, Si and S. Thus, for example “heterocyclic” refers to cyclic (i.e., ring-containing) groups having e.g. N, O, Si or S as part of the ring structure, and having in the range of 3 up to 14 carbon atoms. “Heteroaryl” and “heteroalkyl” moieties are aryl and alkyl groups, respectively, containing e.g. N, O, Si or S as part of their structure. The terms “heteroaryl”, “heterocycle” or “heterocyclic” refer to a monovalent unsaturated group having a single ring or multiple condensed rings, from 1 to 8 carbon atoms and from 1 to 4 hetero atoms selected from nitrogen, sulfur or oxygen within the ring.

As used herein, the term “acrylate” refers to a compound bearing at least one moiety having the structure:

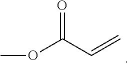

As used herein, the term “methacrylate” refers to a compound bearing at least one moiety having the structure:

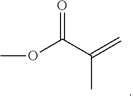

As used herein, the term “free radical initiator” refers to any chemical species which, upon exposure to sufficient energy (e.g., light, heat, or the like), decomposes into parts, which are uncharged, but every one of such part possesses at least one unpaired electron.

As used herein, the term “coupling agent” refers to chemical species that are capable of bonding to a mineral surface and which also contain polymerizably reactive functional group(s) so as to enable interaction with the adhesive composition.

“Modulus” or “Young's modulus” as used herein, is a measure of the stiffness of a material. Within the limits of elasticity, modulus is the ratio of the linear stress to the linear strain, which can be determined from the slope of a stress-strain curve created during tensile testing.

“Thixotropy” as used herein, refers to the property of a material which enables it to stiffen or thicken in a relatively short time upon standing, but upon agitation or manipulation to change to low-viscosity fluid; the longer the fluid undergoes shear stress, the lower its viscosity. Thixotropic materials are therefore gel-like at rest but fluid when agitated and have high static shear strength and low dynamic shear strength, at the same time.

The present invention is based on the goal of providing a horse hoof repair coating that would be easy to apply, durable, fast curing, non-irritating to the animal, and aesthetically pleasing to the eye.

Easy to apply formulations have been developed to be used as coatings for repairing and or preventing damage to the equine hoof. The light-curable invention formulations comprise:

a) at least one acrylated urethane;

b) at least one adhesion promoter(s);

c) at least one tertiary amine;

d) at least one filler;

e) at least one photoinitiator; and

f) at least one free radical inhibitor.

In certain aspects, the formulations also contain at least one pigment.

In one embodiment of the invention, urethane diacrylate (UDA) can be used as the acrylated urethane of the invention formulations. This material can also be called diurethane diacrylate (DUDA) as well as Di-HEA trimethylhexyl dicarbamate. UDA has the following structure, as a mixture of the 2,2,4- and 2,4,4-isomers.

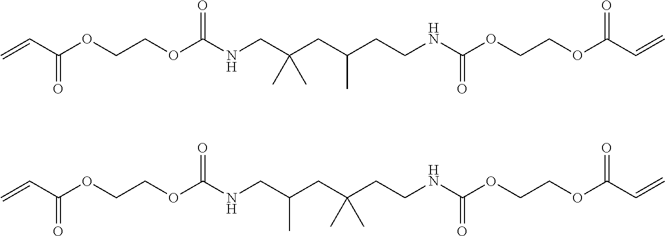

Other alternative acrylated urethanes are also contemplated for use in the practice of the invention. The urethane acrylates that are the most useful in this invention are the aliphatic materials that are liquids at room temperature. Other isocyanates that can be reacted to form curable resins include isophorone diisocyanate, dicyclohexylmethane-4,4′-diisocyanate (H₁₂MDI), hexamethylene diisocyanate (HDI), hexamethylene diisocynate trimer (HDI trimer), hexamethylene diisocyanate biuret (HDI biuret), and hexamethylene diisocyanate uretdione (HDI uretdione). Also contemplated for use in the practice of the invention is the C36 aliphatic dimer diisocyanate (DDI-1410, BASF Corporation). The following structures represent some of the non-limiting acrylated urethane compounds contemplated for use in the practice of the invention:

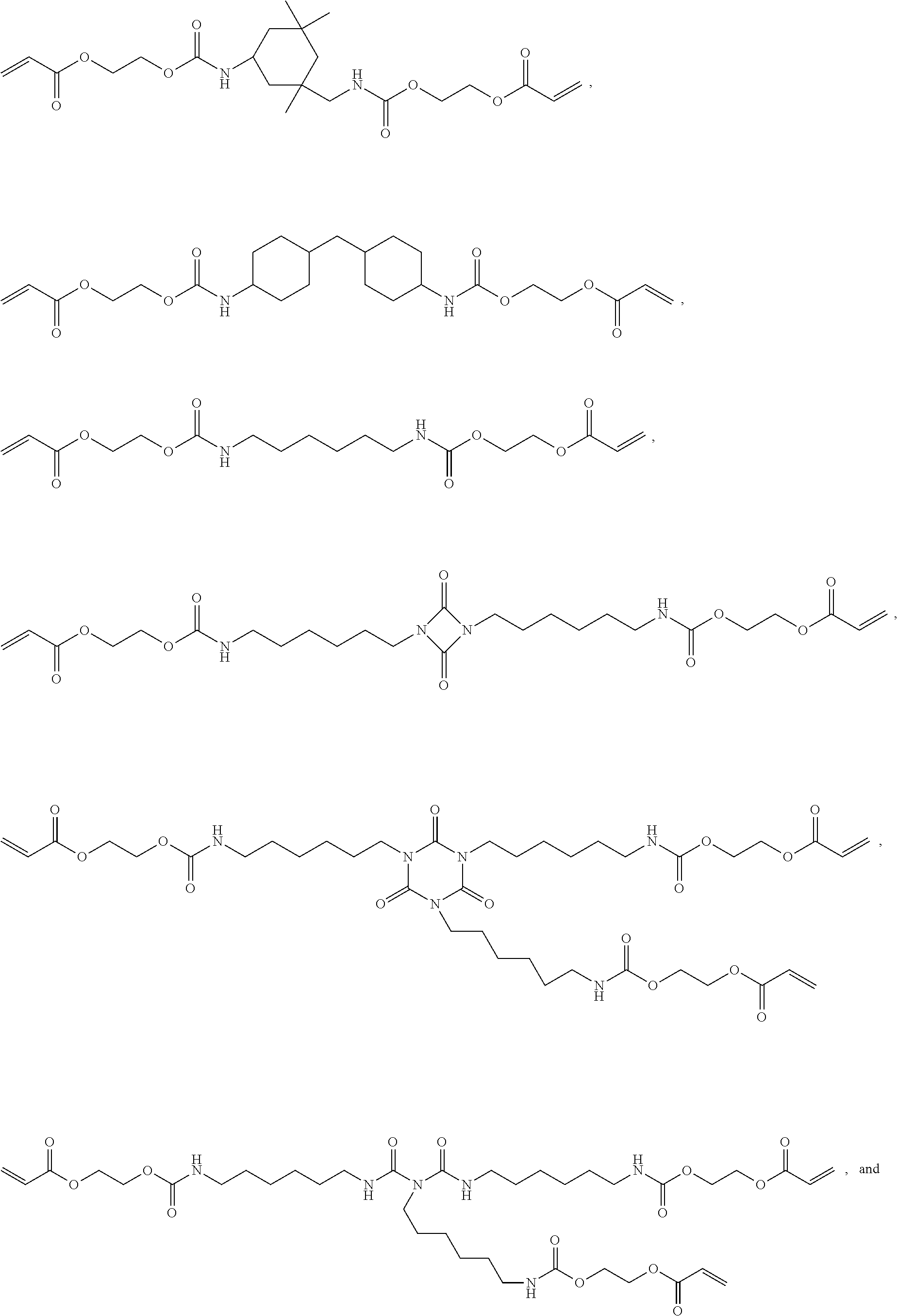

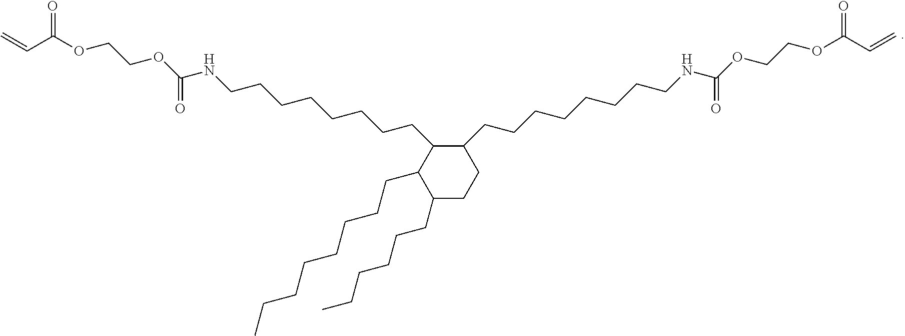 [SMILES: C=CC(=O)OCCOC(=O)NCCCCCCCCC1CCC(CCCCCC)C(CCCCCCCC)C1CCCCCCCCNC(=O)OCCOC(=O)C=C]

The methacrylated urethanes are also contemplated for use in the practice of the invention. The methacrylated urethanes, however, were found to be slow curing. When these materials were used in the invention formulations the coating was still found to be tacky on the surface after exposure to sunlight for over 10 minutes and after one minute of exposure to ultraviolet light (380 nm).

The acrylated urethanes have higher reactivity and cure at a much faster rate; after about one minute exposure to direct sunlight the coatings were found to be non-tacky and produced a hard smooth coating. Upon exposure to UV light the coating were tack-free within 30 seconds.

A small amount of acrylic acid is often an impurity that exists in the starting material hydroxyethyl acrylate. Care must be taken to remove as much of the acrylic acid as possible, since it is known to cause skin irritation.

It is known to those skilled in the art that the all of the acrylated and methacrylated urethane derivatives used in the invention compositions are readily prepared by the reaction of the corresponding isocyanate species with a hydroxyalkylacrylate or hydroxyalkylmethacrylate in the presence of an appropriate catalyst such as dibutyltin dilaurate. Many other metal carboxylates are also used to produce urethanes such as aluminum, bismuth, zinc and zirconium. Tertiary amines such as triethylenediamine, 1,4-diazabicyclo[2.2.2]octane, dimethylcyclohexylamine, and dimethylethanolamine are also used to prepare the acrylated urethanes.

Hydroxyethyl acrylate is the most readily available and lowest cost material for use in price sensitive formulations. However, other material such as hydroxypropyl-, hydroxybutyl-, hydroxypentyl-, and longer chain hydroxyalkyl acrylates are also contemplated for use in the practice of the invention.

In one embodiment of the invention the acrylated urethane is used anywhere from 55 to about 95 wt % based on the total weight of the composition.

In another embodiment of the invention the acrylated urethane is used anywhere from 65 to about 85 wt % based on the total weight of the composition.

Another major component of the invention formulations is 2-hydroxyethyl acrylate phthalate, which has the structure shown below. This material is the reaction product of hydroxyethyl acrylate with phthalic anhydride. This curable carboxylic acid functionalized monomer serves as a good adhesion promoter in the compositions. The adhesion promoter can be used anywhere from 0.5 to about 15% by weight of the total composition.

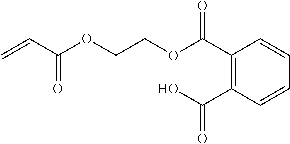 [SMILES: C=CC(=O)OCCOC(=O)c1ccccc1C(=O)O]

In another embodiment of the invention the 2-hydroxyethylacrylate phthalate can be used anywhere from 5 to about 10% by weight of the total composition.

Many inks and paints exhibit thixotropic qualities. It is a desirable characteristic for a fluid to flow sufficiently to form a uniform layer, then to resist further flow, thereby preventing sagging on a vertical surface. In order to get the right thixotropic properties to the formulation a small amount of a filler is added to the formulation.

When tested using a viscometer, a good thixotropic material will ideally have a high viscosity at low RPM's, and as shear force is applied at high RPM the viscosity should drop dramatically. Several fillers were tested in formulations with urethane dimethacrylate from 1-5% by weight to identify those that had the most dramatic affect on thixotropy. The following data summarize the results.

| | | Rheology (cps) | | | |
|---|---|---|---|---|---|
| % UDMA | | 1 rpm | 5 rpm | 10 rpm | 20 rpm |
| | % Filler | | | | |
| 100 | 0 | 7,820 | 7,800 | 7,810 | 7,750 |
| | % Fumed | | | | |
| | Silica | | | | |
| 98.8 | 1.2 | 11,150 | 10,260 | 9,980 | 9,810 |
| 98.8 | 2.0 | 13,460 | 12,240 | 11,820 | 11,420 |
| 94.9 | 5.1 | 26,800 | 22,420 | 21,080 | 19,470 |
| | % Aerosil 200 | | | | |
| 99 | 1.0 | 9,490 | 9,310 | 9,240 | 9,090 |
| 97 | 3.0 | 13,760 | 14,060 | 13,610 | 12,920 |
| 95 | 5.0 | 23,160 | 23,820 | 22,030 | 19,550 |
| | % Aerosil R812 | | | | |
| 98.7 | 1.3 | 10,220 | 9,889 | 9,800 | 9,670 |
| 97.9 | 2.1 | 11,998 | 11,590 | 11,400 | 11,160 |
| 95.0 | 5.0 | 22,634 | 21,940 | 21,796 | 18,980 |
| | % Aerosil R106 | | | | |
| 99.0 | 1.0 | 9,550 | 9,410 | 9,310 | 9,190 |
| 98.0 | 2.0 | 11,350 | 11,180 | 11,040 | 10,770 |
| 95.0 | 5.0 | 25,630 | 21,940 | 23,120 | 20,770 |
| | % Aerosil R972 | | | | |
| 99.0 | 1.0 | 9,220 | 9,270 | 9,220 | 9,160 |
| 98.0 | 2.0 | 10,930 | 10,680 | 10,490 | 10,290 |
| 95.0 | 5.0 | 25,680 | 20,080 | 19,050 | 17,460 |
| | % Aerosil R202 | | | | |
| 99.0 | 1.0 | 11,260 | 9,800 | 9,450 | 9,230 |
| 97.9 | 2.1 | 17,200 | 12,420 | 11,470 | 10,900 |
| 94.9 | 5.1 | 52,490 | 26,600 | 22,020 | 18,880 |
| | % Aerosil 208 | | | | |
| 99 | 1.0 | 11 | 9,680 | 9,170 | 8,910 |
| 97 | 3.0 | 32 | 18,230 | 15,600 | 13,840 |
| 95 | 5.0 | 81 | 34,110 | 26,530 | 21,400 |

The viscosity change from 1 to 5% (w/w) in the silica led to the determination that the Aerosil R202 or Aerosil R208 was the most useful thixotropic agent. Aerosil R208 was judged most economically viable and was used in further material development.

The invention formulations can be cured within ten minutes as the animal is walking around or standing in sunlight, in direct sunlight in about one minute for the clear coatings. With the aid of a UV light the coating cure much faster (in about 30 seconds). To cure the material, a photoinitiator must be added to the formulation.

The photoinitiators contemplated for use in the practice of the invention are those capable of generating free radicals upon exposure to ultraviolet light. BASF, formerly Ciba and many other companies offer a variety of photoinitiators that can be used in the light curing coatings of the invention. All of these photoinitiators are contemplated for use in the practice of the invention.

Several photoinitiators were tested in the application. Some worked much better than did others. Camphorquinone was originally used in the compositions. However, the cured coating would often appeared to have a greenish-blue tint and this was thought to be aesthetically unappealing.

IRGACURE® 4265, which is a liquid photoinitiator composed of a combination of 2-hydroxy-2-methyl-1-phenyl 1-propanone and diphenyl (2, 4, 6-trimethylbenzoyl) phosphine oxide was found to be an effective photoinitiator in the formulation. Ethyl(2,4,6-trimethylbenzoyl)phenyl phosphonate commonly known as IRGACURE® TPO-L (structure shown below) was found to be an ideal photoinitiator for this type of formulation. Because of its absorption behavior in the long-wave range of the UV spectrum, it is preferably used to cure pigmented UV curable coatings and surfaces as well as UV stabilized coatings. The amount of photoinitiator contemplated for use in the practice of the invention can vary from 3 to about 10% by weight of the total formulation.

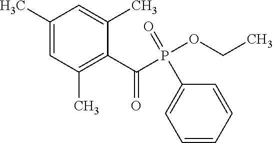 [SMILES: CCOP(=O)(C(=O)c1c(C)cc(C)cc1C)c1ccccc1]

The solid form of IRGACURE® TPO; 2,4,6-trimethylbenzoyldiphenyl phosphine oxide (structure shown below) also serves as a UV initiator in the formulation. The advantage of the solid form of TPO is that it provides for the measurement of a melting point to confirm continued stability over time. The liquid TPO form does not provide this stability confirmation, although the formulation is easier to achieve when the liquid form is used.

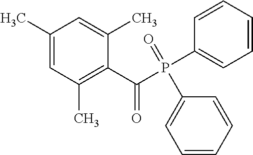 [SMILES: Cc1cc(C)c(C(=O)P(=O)(c2ccccc2)c2ccccc2)c(C)c1]

Many of the free-radial generating photoinitiators work synergistically in the presence of a tertiary amine compound. It has been found that the tertiary amines that are most affective in radical polymerization are the ones that contain at least one aromatic group. Primary and secondary amines often act as inhibitors in the free-radical reaction. Ethyl-4-dimethylaminobenzoate (structure shown below) was found to be a tertiary amine that was very affective in the light cured formulation. The invention formulations however are not limited to only this compound; many other tertiary aromatic amine compounds are also contemplated for use in the practice of the invention. The amount of tertiary aromatic amine can range from 0.1 to about 10% by weight of the formulation.

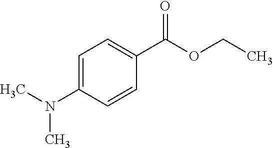

Since the invention formulations are free-radically curable materials, they must also contain free-radical inhibitors. The addition of free-radical inhibitors provides a suitable product shelf life and prevents premature curing of the material. Many of these free-radical inhibitors are known as antioxidants. These compounds include reducing agents such as thiols, ascorbic acid and polyphenols. In the laboratory, phenolic compounds such as butylated hydroxytoluene (BHT) and methoxyhydroquinone (MEHQ), hydroquinone (HQ), are added to formulations. The combination of a phenolic inhibitor with benzoquinone derivatives has a synergistic effect and is a more potent inhibitor mix. The addition of certain nitrosyl compounds, certain tertiary amines, nitro-aromatic compounds are known to those skilled in the art to prevent premature free radical curing. The amount of free radical inhibitor contemplated for use in the practice of the invention can vary from 50 ppm (parts per million) to about 5000 ppm.

Additional ingredients that can be added to the formulations of the invention include compounds such as hydroxyethyl acrylate. Not to be bound by any particular theory, but such compounds are often added to boost the adhesion of the material to various substrates or to modify the viscosity of the formulation.

Also contemplated in the practice of the invention is the addition of colorants, such as dyes and pigments. The addition of these types of compounds to the formulations create a more aesthetically pleasing coating for the benefit of the animal and the owner. The following colorants and dyes are suitable for use in the formulations of the invention.

Iron Oxides.

These inorganic pigments are used in all types of cosmetics and have three basic shades: black, yellow and red. By blending these three oxides in the right proportions you can produce an array of browns, tans, and ambers. By careful blending an array of natural looking flesh tones may be produced.

Chromium Dioxides.

The chromium dioxides are used for most categories of cosmetic preparations. Colors range from dull olive green, to a blue green, or bright green.

Ultramarines.

Ultramarines vary in shade from bright blue to violet, pink and even green. These pigments are also based on lazurite various shades and are contemplated for use in the practice of the invention.

Manganese Violet.

This brightly colored inorganic pigment is purple colored, based on ammonium manganese diphosphate.

Iron Blue.

This very deep intense dark blue pigment is widely used in all cosmetic applications. Ferric ammonium ferrocyanide and lazurite are contemplated for use in making blue colored product.

White Pigments.

White pigments are widely used in all cosmetics, they have extremely good covering power and are almost totally inert in addition to being extremely stable to heat and light. Titanium Dioxide and Zinc Oxide are the most commonly used in cosmetics. Other known white pigments contemplated for use in the practice of the invention include but are not limited to compounds such as: aluminum; aluminum hydroxide sulphate; aluminum silicate; barium sulphate; bismuth chloride oxide; calcium carbonate; calcium sulphate; magnesium carbonate; aluminum stearate; magnesium stearate; zinc stearate; and calcium stearate.

Mica.

Chemically Mica is potassium aluminum silicate dihydrate. It is mined as the ore muscovite and occurs in multi-layered bright translucent sheets.

Black.

The color black is typically derived from carbon black and is also contemplated for use in the practice of the invention.

In certain embodiments of the invention, one or more of the the following pigments are used:

Blue: Iridescent Blue (Peacock) CP-6046

Pink: Neon Pink Red CP-26

Black: Black Pigment CP-89003

White: Titanium dioxide

Blue: Ultramarine Blue

Red: D&C Red 7 Calcium Lake 70%.

In addition to inorganic pigments, there are numerous organic dyes that are also contemplated for use in the practice of the invention. These materials are available in various colors and are known to those skilled in the art.

### EXAMPLES

### Materials and Methods

The hoof repair formulations were brushed on to the hoof wall of a horse after cleaning the hoof of the animal. The clear coatings were formulated to form a hard tack-free coating upon exposure to direct sunlight in about a minute. The formulations were cured by exposure to a UV flashlight (380 nm) for 30 seconds per illuminated areas. For the hoof repair coatings that contain pigments, the illumination time was increased to approximately one minute exposure to the UV flashlight per illuminated areas. The material is prophylactic and regular treatment of the formulation on the hoof wall prevents crack formation.

### Example 1

A hoof repair composition was prepared according to the following formulation:

| Component | Amount (wt %) |
|---|---|
| Urethane Diacrylate | 90.2% |
| Silica R208 | 1.5% |
| Camphorquinone | 2.9% |
| Ethyl 4-dimethylamino Benzoate | 2.9% |
| 2-Hydroxyethyl Acrylate Phthalate | 1.0% |
| Butylated Hydroxytoluene | 1.5% |

Rheology data at 25° C. were measured as follows:

| Speed | Viscosity |
|---|---|
| 1 RPM | 13,298 cps |
| 5 RPM | 7,782 cps |
| 10 RPM | 6,681 cps |
| 20 RPM | 5,944 cps |

The Thixotropic Index, (1 RPM viscosity)/(10 RPM viscosity) was 1.99

This material cured upon 60 seconds exposure to direct sunlight, forming a tack-free coating.

### Example 2

A hoof repair composition was prepared according to the following formulation:

| Component | Amount (wt %) |
|---|---|
| Urethane Diacrylate | 89.915% |
| Silica R208 | 3.5% |
| Camphorquinone | 3% |
| Ethyl 4-dimethylamino Benzoate | 3% |
| 2-Hydroxyethyl Acrylate Phthalate | 0.5% |
| Butylated Hydroxytoluene | 0.085% |

Rheology data at 25° C. were measured as follows:

| Speed | Viscosity |
|---|---|
| 1 RPM | 32,925 cps |
| 5 RPM | 13,758 cps |
| 10 RPM | 10,316 cps |
| 20 RPM | 8,276 cps |

The Thixotropic Index, (1 RPM viscosity)/(10 RPM viscosity) was 3.19.

This material cures upon 30 seconds exposure to direct sunlight, forming a tack-free coating.

### Example 3

A hoof repair composition was prepared according to the following formulation:

| Component | Amount (wt %) |
|---|---|
| Urethane Diacrylate | 77.1% |
| Silica R208 | 0.9% |
| Irgacure 4265 | 6% |
| Ethyl 4-dimethylamino Benzoate | 6% |
| 2-Hydroxyethyl Acrylate Phthalate | 10.0% |
| Butylated Hydroxytoluene | 0.0367% |

Rheology data at 25° C. were measured as follows:

| Speed | Viscosity |
|---|---|
| 1 RPM | 4,529 cps |
| 5 RPM | 3,508 cps |
| 10 RPM | 3,298 cps |
| 20 RPM | 3.128 cps |

Thixotropic Index, (1 RPM viscosity)/(10 RPM viscosity)=1.37

This material cures upon 30 seconds exposure to the UV flashlight at 380 nm, forming a tack-free coating.

### Example 4

A hoof repair composition was prepared according to the following formulation:

| Component | Amount (wt %) |
|---|---|
| Urethane Diacrylate | 70.0% |
| 2-Hydroxyethyl Acrylate Phthalate | 10.0% |
| TPO solid | 7.5% |
| Ethyl 4-dimethylamino Benzoate | 5% |
| 2-Hydroxyethyl Acrylate | 5% |
| Silica R208 | 1.75% |
| Butylated Hydroxytoluene | 0.235% |

Rheology data at 25° C. were measured as follows:

| Speed | Viscosity |
|---|---|
| 1 RPM | 5,478 cps |
| 5 RPM | 4,325 cps |
| 10 RPM | 4,046 cps |
| 20 RPM | 3,751 cps |

Thixotropic Index, (1 RPM viscosity)/(10 RPM viscosity)=1.35

This material cured upon 30 seconds exposure to the UV flashlight at 380 nm, forming a tack-free coating.

### Example 5

A hoof repair composition was prepared according to the following formulation:

| Component | Amount (wt %) |
|---|---|
| Isophorone Urethane Diacrylate | 60.5% |
| 2-Hydroxyethyl Acrylate | 15.0% |
| 2-Hydroxyethyl Acrylate Phthalate | 10.0% |
| TPO solid | 7.5% |
| Ethyl 4-dimethylamino Benzoate | 5.0% |
| Silica Aerosil 200 | 1.75% |
| Butylated Hydroxytoluene | 0.235% |

Rheology data at 25° C. were measured as follows:

| Speed | Viscosity |
|---|---|
| 1 RPM | 3,760 cps |
| 5 RPM | 3,663 cps |
| 10 RPM | 2,863 cps |
| 20 RPM | 2,667 cps |

The Thixotropic Index, (1 RPM viscosity)/(10 RPM viscosity) was 1.31

This material cures upon 30 seconds exposure to the UV flashlight at 380 nm, forming a tack-free coating.

### Example 6

A hoof repair composition was prepared according to the following formulation:

| Component | Amount (wt %) |
|---|---|
| Isophorone Urethane Diacrylate | 59.8% |
| 2-Hydroxyethyl Acrylate | 15.0% |
| 2-Hydroxyethyl Acrylate Phthalate | 10.0% |
| TPO solid | 7.5% |
| Ethyl 4-dimethylamino Benzoate | 5.0% |
| Silica Aerosil 200 | 2.5% |
| Butylated Hydroxytoluene | 0.235% |

Rheology data at 25° C. were measured as follows:

| Speed | Viscosity |
|---|---|
| 1 RPM | 6,372 cps |
| 5 RPM | 4,597 cps |
| 10 RPM | 4,185 cps |
| 20 RPM | 3,717 cps |

The Thixotropic Index, (1 RPM viscosity)/(10 RPM viscosity) was 1.52

This material cures upon 30 seconds exposure to the UV flashlight at 380 nm, forming a tack-free coating.

### Example 7

A hoof repair composition was prepared according to the following formulation:

| Component | Amount (wt %) |
|---|---|
| Isophorone Urethane Diacrylate | 62.5% |
| 2-Hydroxyethyl Acrylate | 13.0% |
| 2-Hydroxyethyl Acrylate Phthalate | 10.0% |
| TPO solid | 7.5% |
| Ethyl 4-dimethylamino Benzoate | 5.0% |
| Silica Aerosil 200 | 1.75% |
| Butylated Hydroxytoluene | 0.235% |

Rheology data at 25° C. were measured as follows:

| Speed | Viscosity |
|---|---|
| 1 RPM | 5,448 cps |
| 5 RPM | 4,671 cps |
| 10 RPM | 4,335 cps |
| 20 RPM | 3,920 cps |

The Thixotropic Index, (1 RPM viscosity)/(10 RPM viscosity) was 1.26

This material cures upon 30 seconds exposure to the UV flashlight at 380 nm, forming a tack-free coating.

### Example 8

A hoof repair composition was prepared according to the following formulation:

| Component | Amount (wt %) |
|---|---|
| Isophorone Urethane Diacrylate | 62.0% |
| 2-Hydroxyethyl Acrylate | 13.0% |
| 2-Hydroxyethyl Acrylate Phthalate | 10.0% |
| TPO solid | 7.5% |
| Ethyl 4-dimethylamino Benzoate | 5.0% |
| Silica Aerosil 200 | 2.3% |
| Butylated Hydroxytoluene | 0.235% |

Rheology data at 25° C. were measured as follows:

| Speed | Viscosity |
|---|---|
| 1 RPM | 6,295 cps |
| 5 RPM | 5,305 cps |
| 10 RPM | 4,792 cps |
| 20 RPM | 4,226 cps |

Thixotropic Index, (1 RPM viscosity)/(10 RPM viscosity)=1.31

This material cures upon 30 seconds exposure to the UV flashlight at 380 nm, forming a tack-free coating.

### Example 9

A black hoof repair composition was prepared according to the following formulation:

| Component | Amount (wt %) |
|---|---|
| Isophorone Urethane Diacrylate | 59.8% |
| 2-Hydroxyethyl Acrylate | 12.6% |
| 2-Hydroxyethyl Acrylate Phthalate | 10.0% |
| TPO solid | 9.2% |
| Ethyl 4-dimethylamino Benzoate | 5.4% |
| Silica Aerosil 200 | 1.6% |
| Black Pigment | 1.2% |
| Butylated Hydroxytoluene | 0.2% |

Rheology data at 25° C. were measured as follows:

| Speed | Viscosity |
|---|---|
| 1 RPM | 3,963 cps |
| 5 RPM | 3,567 cps |
| 10 RPM | 3,456 cps |
| 20 RPM | 3,251 cps |

The Thixotropic Index, (1 RPM viscosity)/(10 RPM viscosity) was 1.15

This material cured upon 60 seconds exposure to the UV flashlight at 380 nm, forming a tack-free coating.

### Example 9

### Blue Glitterati

A Blue Glitter hoof repair composition was prepared according to the following formulation:

| Component | Amount (wt %) |
|---|---|
| Urethane Diacrylate | 63.7% |
| 2-Hydroxyethyl Acrylate Phthalate | 10.2% |
| Silver Glitter | 7.5% |
| TPO solid | 7.2% |
| Ethyl 4-dimethylamino Benzoate | 4.4% |
| 2-Hydroxyethyl Acrylate | 5% |
| Silica R208 | 1.1% |
| Blue Pigment | 0.7% |
| Butylated Hydroxytoluene | 0.2% |

This material cured upon 60 seconds exposure to the UV flashlight at 380 nm, forming a tack-free coating.

The rheological characteristics could not be determined in the rheometer as the glitter content interferes with the measurement.

### Example 10

### Too Haute to Trot Pink

A pink hoof repair composition was prepared according to the following formulation:

| Component | Amount (wt %) |
|---|---|
| Urethane Diacrylate | 56.1% |
| Silver Glitter | 11.9% |
| 2-Hydroxyethyl Acrylate Phthalate | 10.2% |
| TPO solid | 8.9% |
| Ethyl 4-dimethylamino Benzoate | 5.4% |
| 2-Hydroxyethl Acrylate | 5% |
| Silica R208 | 1.3% |
| Pink Pigment | 1.0% |
| Butylated Hydroxytoluene | 0.2% |

This material cured upon 60 seconds exposure to the UV flashlight at 380 nm, forming a tack-free coating.

The rheological characteristics could not be determined in the rheometer as the glitter content interfered with the measurement.

What is claimed is:

1. A protective, lightweight, curable hoof composition for ungulate mammals comprising:
a) at least one acrylated urethane;
b) at least one adhesion promoter(s);
c) at least one tertiary amine;
d) at least one filler;
e) at least one photoinitiator; and
f) at least one free radical inhibitors,
wherein the composition is suitable for application to the hoof of ungulate animals.

2. The composition of claim 1, further comprising at least one pigment.

3. The composition of claim 1, wherein the at least one acrylated urethane is selected from the group consisting of any one of the following structures:

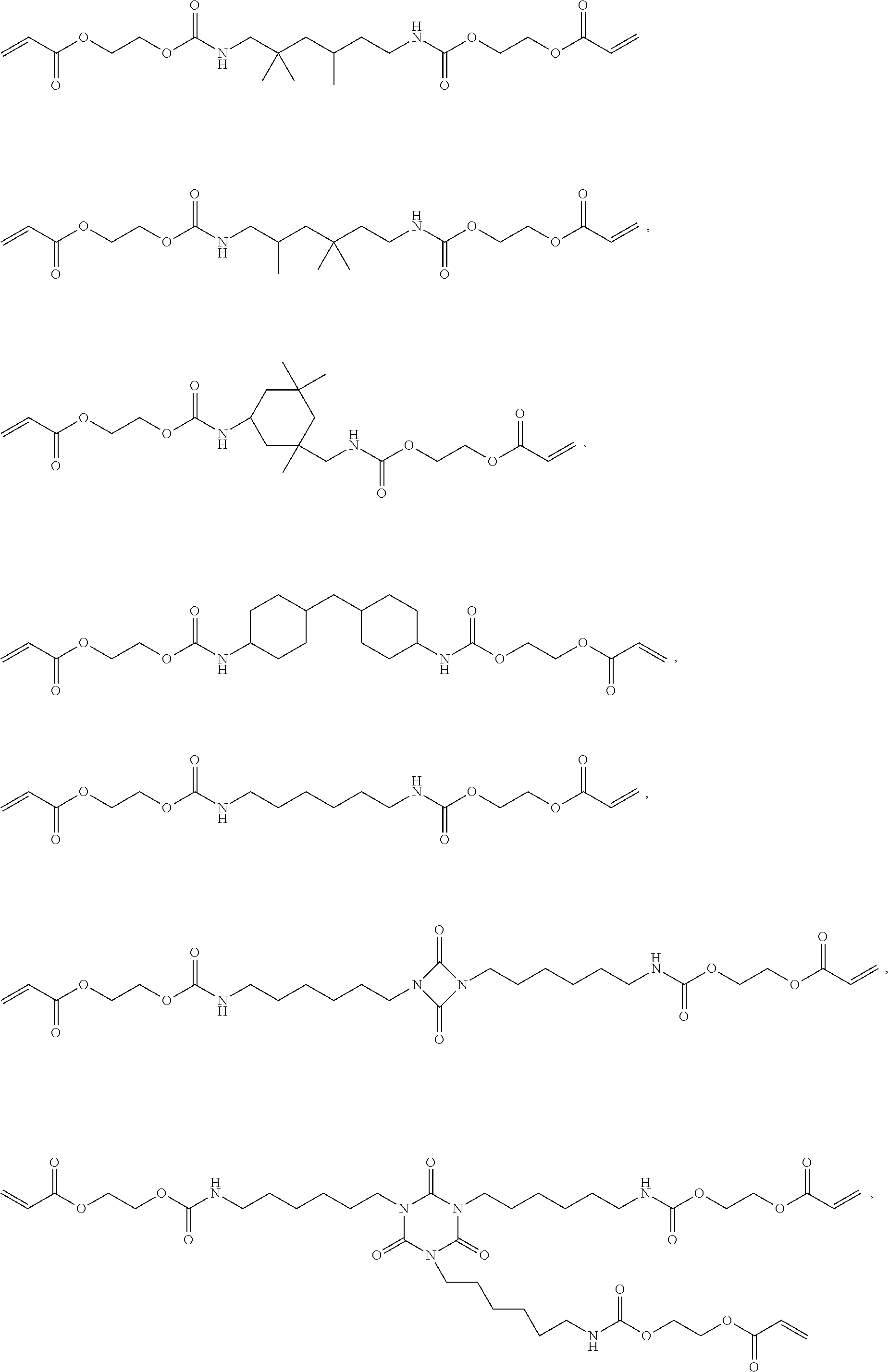

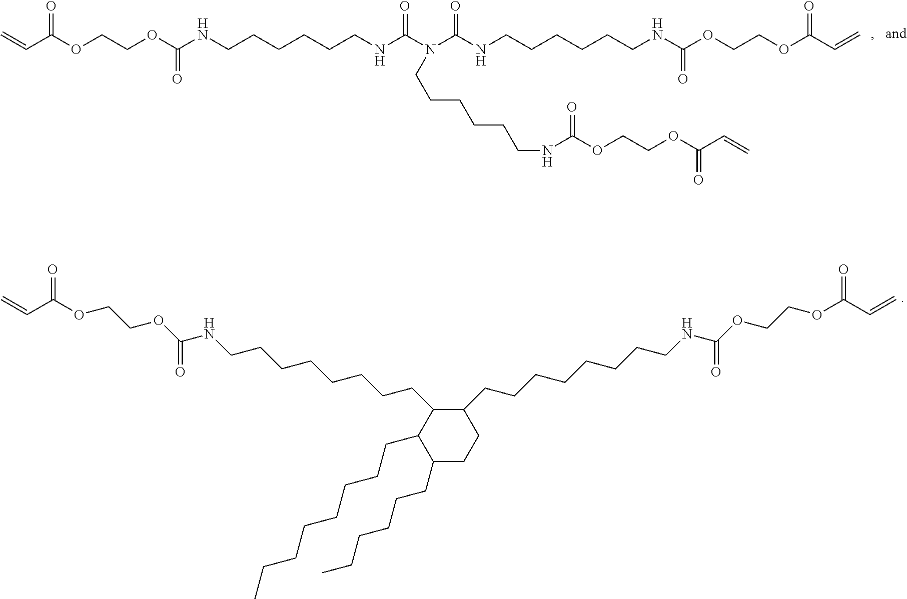

4. The composition of claim 1, wherein the at least one acrylated urethane comprises about 55 to about 95 weight percent (wt %) based on the total weight of the composition.

5. The composition of claim 1, wherein the at least one acrylated urethane comprises about 65 to 85-wt % based on the total weight of the composition.

6. The composition of claim 1, wherein the at least one adhesion promoter is 2-hydroxyethyl acrylate phthalate.

7. The composition of claim 1, wherein the at least one adhesion promoter is 2-hydroxyethyl acrylate.

8. The composition of claim 1, wherein the at least one adhesion promoter comprises 0.5 to about 30 wt % based on the total weight of the composition.

9. The composition of claim 1, wherein the at least one adhesion promoter comprises 10 to about 25 wt % based on the total weight of the composition.

10. The composition of claim 1, wherein the at least one filler comprises silica.

11. The composition of claim 1, wherein the at least one filler comprises 0.9 to about 3.5 wt % based on the total weight of the composition.

12. The composition of claim 1, wherein the at least one photoinitiator comprises ethyl(2,4,6-trimethylbenzoyl)phenyl phosphonate.

13. The composition of claim 1, wherein the at least one photoinitiator comprises 2,4,6-trimethylbenzoyldiphenyl phosphine oxide.

14. The composition of claim 1, wherein the at least one photoinitiator comprises 3 to about 10 wt % based on the total weight of the composition.

15. The composition of claim 1, wherein the at least one tertiary amine comprises ethyl 4-dimethylaminobenzoate.

16. The composition of claim 1, wherein the at least one tertiary amine comprises 2 to about 6 wt % based on the total weight of the composition.

17. The composition of claim 1, wherein the at least one free radical inhibitor comprises butylated hydroxytoluene.

18. The composition of claim 1, wherein the composition is thixotropic.

19. The composition of claim 1, wherein the composition cures to a hard, tack-free coating upon exposure to direct sunlight or ultraviolet light after brushing onto the surface of the hoof of the ungulate.

20. A method of protecting the hoof of an ungulate animal comprising the steps of:
(a) providing the composition of claim 1;
(b) brushing the composition on the surface of the hoof of the ungulate; and
(c) exposing the composition to direct sunlight or ultraviolet light;
wherein the composition cures to a hard, tack-free coating upon exposure to direct sunlight or ultraviolet light, thereby protecting the hoof of an ungulate animal.
